(19) United States
(12) Patent Application Publication
(10) Pub. No.: US 2016/0220811 A1
(43) Pub. Date: Aug. 4, 2016

(54) RIGHT ANGLE CANNULA PROBE FOR CORONARY SINUS CANNULATION

(71) Applicant: THE TRUSTEES OF COLUMBIA UNIVERSITY IN THE CITY OF NEW YORK, NEW YORK, NY (US)

(72) Inventors: Henry M. Spotnitz, New York, NY (US); Keith Yeager, Jersey City, NJ (US)

(21) Appl. No.: 15/095,959

(22) Filed: Apr. 11, 2016

Related U.S. Application Data
(63) Continuation of application No. PCT/US14/60114, filed on Oct. 10, 2014.
(60) Provisional application No. 61/889,937, filed on Oct. 11, 2013.

(51) Int. Cl. A61N 1/05

(52) U.S. Cl. CPC A61N 1/0587; A61N 1/056; A61N 2001/0585; A61B 2017/00526

(57) ABSTRACT

A medical device comprising a cannula probe for insertion into a patient with a proximal end and a distal end, a lumen extending between the proximal and distal ends, and a portion of the cannula probe configured with a contour customized to a patient's anatomy. The distal end of the cannula probe is disposed at a substantially right angle, with at least the distal tip of the cannula probe formed of a rigid material sufficient to maintain the substantially right angle orientation.

### CROSS REFERENCE TO RELATED APPLICATIONS

This application is a continuation of co-pending PCT/US14/60114, filed Oct. 10, 2014, which claims the benefit of U.S. Provisional Application No. 61/889,937 filed Oct. 11, 2013, the entirety of each of which is hereby incorporated by reference.

### BACKGROUND OF THE DISCLOSED SUBJECT MATTER

### Field of the Disclosed Subject Matter

More than 500,000 new cases of congestive heart failure (CHF) occur annually in the United States. CHF can lead to left-ventricle (LV) systolic dysynchrony through regional myocardial weakness, scarring and conduction deficits. In clinical trials, cardiac resynchronization therapy (CRT) has reversed dysynchrony by pacing opposing LV wall segments from both the RV apex and lateral branch of the coronary sinus (CS). CRT has become the standard of care for selected patients with advanced CHF, LV dysfunction, and intraventricular conduction delay. “Permanent” CRT reduces LV size and mitral regurgitation, improves function, and decreases morbidity and mortality. However, the nonresponse rate for CRT is 30% or more. The estimated CRT population is approaching 100,000 annually in the United States and is extending into Class II CHF.

Standard CRT is implemented with endocardial leads in the right atrium, right ventricle and left ventricle (RA, RV, and LV). CS cannulation for endocardial LV lead insertion is technically difficult and fails in 8-13% of attempts. Technical difficulty includes precisely steering flexible catheters over 50 cm in length. Steerable catheters are helpful, but leverage is often inadequate to reach far posterior or angulated CS locations. The CS is radiolucent, and its precise location is variable. Late stage coronary arteriography or dye pulses into the RA can help localize the CS but also can be problematic for volume-overloaded patients with marginal renal function. Fatemi et al averaged 43±29 minutes to place a CS lead, and LV lead implant time exceeded 72 minutes in 16% of patients [See, Fatemi M, Etienne Y, Gilard M, et al. Short and long-term single-centre experience with an S-shaped unipolar lead for left ventricular pacing. Europace 5:207-11, 2003].

When the CS cannot be cannulated, alternatives include abandoning CRT or referring patients for epicardial lead insertion via formal, mini, or “robotic” thoracotomy. Problems with traditional thoracotomy devices and techniques include limited LV access, hemodynamic instability, dense scarring, arrhythmias, and bleeding. Pericardial adhesions develop after cardiac surgery or myocardial infarction. Right parasternal mediastinotomy (RPSM), originally developed for biopsies in the anterior mediastinum, provides greater mechanical advantage, better control of approach angles, and spatial mobility. RPMS can also promote sensor introduction, adjustment of lead location, and dye injection for venous angiography. Short distances from the RA appendage to the CS will promote leverage for lead advancement. RPMS can also promote exchanges of LV lead types and implant location.

RPSM has been used for pacemaker insertion, but CS cannulation for CRT has not been reported. The device and methods disclosed herein is based on obturator and cannula designs for vascular access/pacemaker insertion using the Seldinger technique. Additionally, the disclosed device can be incorporated with curving Teflon cannula probes, with microwave heating and obturators with incorporation of copper wires, or combinations thereof.

A large and variety of steerable guidewires, curved cannulas, and over-the-wire leads compose the current standard equipment for CS lead insertion from the subclavian or jugular veins. However, these do not provide sufficient control for reliable CS cannulation in enlarged hearts. In contrast, the CS is routinely cannulated for cardioplegia administration in less than a minute during cardiac surgery.

In accordance with an aspect of the disclosed system, obturators cannulas, and in some instances guidewires, can contain sensors (e.g. pressure, flow, temperature, oxygen and audio sensors) to help localize the CS. The CS leads disclosed herein have been successfully implanted through cardioplegia cannulas during thoracotomy in mammals, e.g., pigs. Similarly, pressure, oximetry, and temperature measurements in the CS have been achieved using the system, devices and methods disclosed herein.

Additionally, the system and method disclosed herein allows for LV lead insertion within 20 minutes of initial skin incision with a failure rate of 0%, vs. 43 minutes for prior art techniques (See Fatemi et al.) and 10-13% failure rates. Lead insertion by thoracotomy with epicardial leads in the presence of adhesions can take many hours, with bleeding, mortality, and morbidity risks and prolonged and expensive hospitalization. RPSM, as disclosed herein, also facilitate RA and RV lead insertion, reducing total operating time to less than one hour.

### SUMMARY OF THE DISCLOSED SUBJECT MATTER

In accordance with an aspect of the present disclosure, a medical device comprises a cannula probe for insertion into a patient having a proximal end and a distal end defining a longitudinal length therebetween, a lumen extending between the proximal and distal ends, wherein at least a portion of the cannula probe is configured with a contour customized to a patient's anatomy, and wherein the distal end of the cannula probe is disposed at a substantially right angle, with at least the distal tip of the cannula probe formed of a rigid material sufficient to maintain the substantially right angle orientation.

In one embodiment, the substantially right angle at the distal tip extends 0.75 inches from the midline of the device and has a radius of curvature of 0.75 inches.

Additionally, the medical device can include an outer sheath having a flexible hollow shaft, with the cannula probe disposed inside the outer sheath. Also, a line of weakness can extend along at least a portion of the longitudinal length of the cannula probe. In some embodiments a guidewire is disposed within the lumen of the cannula probe, with the cannula probe circumferentially extending a distance less than the circumference of the guidewire.

Also, a retention piece can be included which is slidingly received by the cannula probe. In some instances, the retention piece is configured with a slanted distal end such that the slanted distal end of the retention piece radially displaces the guidewire out of the cannula probe lumen upon axial movement of the retention piece. The retention piece can be formed with radially inner and outer edges with a larger circumferential width than a middle portion disposed between the radially inner and outer edges such that the radially inner portion of the retention piece overlies a portion of an inner surface of the cannula probe. Additionally, the radially inner outer edge of the retention piece can be circumferentially aligned with an outer surface of the cannula probe. Some embodiments include at least one sensor, disposed at the distal tip of the cannula probe, and/or guidewire.

In accordance with another aspect of the disclosure, a medical kit is disclosed comprising: a cannula probe for insertion into a patient having a proximal end and a distal end defining a longitudinal length therebetween, a lumen extending between the proximal and distal ends, a sensor disposed at the distal end of the cannula probe, wherein at least a portion of the cannula probe is configured with a contour customized to a patient's anatomy, and wherein the distal end of the cannula probe is disposed at a substantially right angle, with at least the distal tip of the cannula probe formed of a rigid material sufficient to maintain the substantially right angle orientation; and a haptic feedback device, the haptic feedback device configured to receive a signal from the sensor.

In some embodiments, the haptic feedback device includes a vibratory feedback mechanism, in others an auditory feedback mechanism. The haptic feedback device and cannula probe can be configured as separate components, and include a transmitter for wireless communication with the sensor disposed at the distal tip of the cannula probe.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of a cannula probe contour design and optimization technique in accordance with the disclosed subject matter.

FIGS. 2-3 are exemplary depictions of the CT based modeling technique in accordance with the disclosed subject matter.

FIG. 4 is a representative graph of the time savings during procedures employing the device and methods disclosed herein.

FIGS. 5A-B are representative images of the device disclosed herein disposed within a subject.

FIGS. 6-14 are exemplary depictions of various embodiments of the device of the disclosed subject matter.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

The system and methods disclosed herein is particularly applicable to patients who fail endocardial lead insertion and are referred to cardiothoracic surgeons for epicardial lead insertion. Examples of such epicardial leads are disclosed in copending U.S. application Ser. No. 13/381,046, the entirety of which is hereby incorporated by reference. The device disclosed herein is particularly appealing to surgeons planning operations on patients who have undergone multiple open operations for acquired and/or congenital heart disease. Dense adhesions in the pericardial space of such patients obscure anatomical landmarks and create risk of cardiac or vascular injury as well as bleeding resulting from division of adhesions to expose the epicardium. Implanting an epicardial LV lead in such a setting is daunting, and myocardial fibrosis can require many sites to be tested before adequate electrical properties are secured. Implanting LV leads is tedious and dangerous under these circumstances, long-term lead utility is unpredictable, and risks of infection, intraoperative hypotension and arrhythmias are substantial. The device and techniques disclosed herein address the difficulty of surgery under these circumstances.

The spectrum of patients referred for lead insertion by thoracotomy ranges from a long surgical history and extensive pleuropericardial adhesions to patients with no adhesions and no previous surgery or myocardial infarction. For the simpler group, minithoracotomy and robotic approaches are currently available. However, endocardial leads are generally regarded as more reliable for long-term use, in part because of high flexion stresses and ongoing fibrosis at the epicardial surface. The system and method disclosed herein utilizes a mini-thoracotomy to expose the heart, but the implanted lead itself is equivalent to standard endocardial leads in design and intracardiac location of the electrical contact surface. Performance of leads implanted in this way is at least equivalent to the best endocardial leads and superior to epicardial leads.

Additionally, the system and method disclosed herein enhances both safety and efficacy, thereby increasing the availability and applicability of epicardial CRT procedures. The number of patients currently referred for epicardial lead insertion is far less than 10-13% of attempted endocardial implants. By employing the system and method disclosed herein many more patients failing endocardial insertion are eligible for subsequent procedures since the risk and invasiveness of epicardial approaches can be reduced.

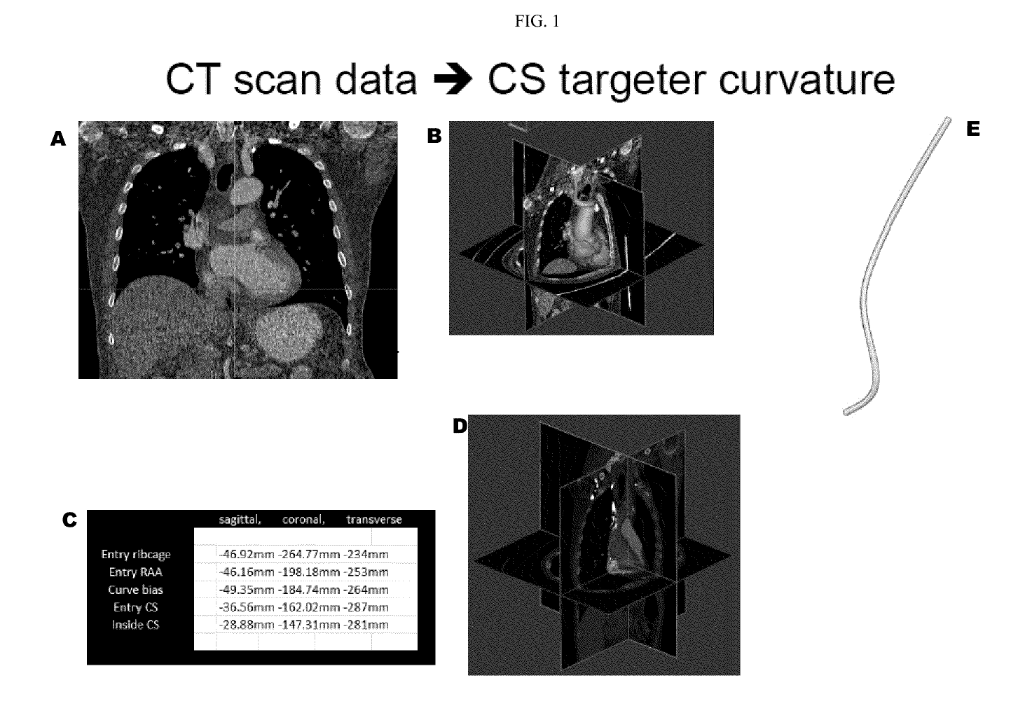

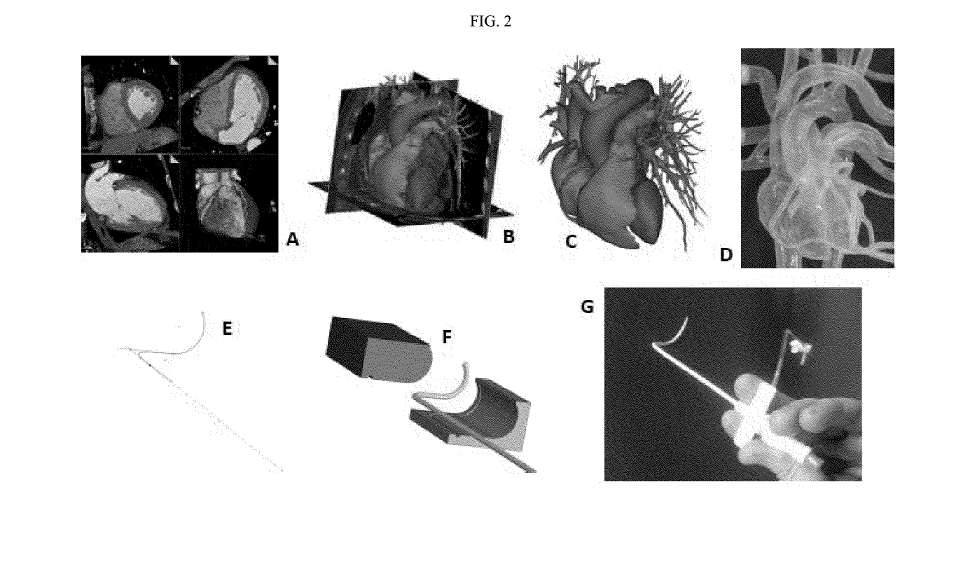

In accordance with an aspect of the disclosed subject matter, the cannula and/or cannula probe is customized for entry into the CS from RPSM. The customization includes distinct curvatures and optimized length. An exemplary representation of this customization procedure is depicted in FIGS. 1-3. Referring to FIGS. 1A to 1D an exemplary embodiment of the process for manufacturing a cannula and/or cannula probe customized for a entry to a particular patient's CS. In regard to FIGS. 1A to 1D, the process employs CT scan data (FIG. 1A) to design a CS targeter curvature that is customized to a specific patient's anatomy. The system and method provide a streamlined technique for processing patient datasets by reducing the targeter definition or modeling inputs to five select points or locations, as shown in FIG. 1B. In the embodiment shown in FIG. 1B, the five select points for modeling are: 1) Entry ribcage: 2) Entry RAA; 3) Curve bias; 4) Entry CS; 5) Inside CS. The sagittal, coronal and transverse measurements are taken at these five locations. Referring to FIGS. 1C and 1D, automated generation of cannula probe geometry (FIG. 1E) is achieved utilizing the five key points derived from the patient CT scan. Accordingly, 3D segmentation is no longer necessary.

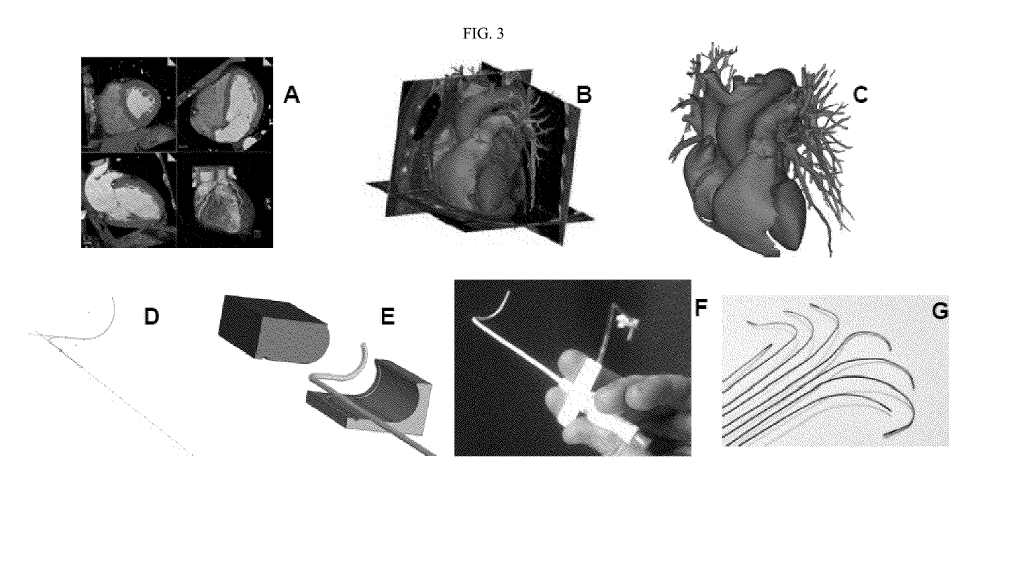

FIGS. 2A-G depict exemplary stages in the design of the anatomically based obturator/cannula probe geometry. First, a CT scan (as shown in FIG. 2A) is captured and imported into a software model (e.g. Mimics) and a 3D volume is fit to the data (as shown in FIG. 2B). This 3D data is inputted in solid modeling software (e.g. Solidworks), as shown in FIG. 2C. Then a physical model of the patient anatomy is rapid prototyped (as shown in FIG. 2D) for benchtop testing, and an anatomic path is extracted (as shown in FIG. 2E) and used to design and fabricate a mold (as shown in FIG. 2F) which shapes a cannula probe and obturator to the desired geometry (as shown in FIG. 2G). This customized geometric design is tailored to the patient's anatomy and will facilitate insertion through the right atrium into the coronary sinus. Likewise, FIGS. 3A-G depict a similar process for designing and fabricating anatomically based obturator/cannula probe geometry, in which multiple data sets can be collected and used to design a family of curved cannula probes (as shown in FIG. 3G).

Additionally, the obturators guiding the cannulas incorporate bendable guidewires and sensor technologies. In some embodiments the guidewires are made of copper or nitinol, or other suitable material which exhibits sufficient rigidity to maintain the customized curvature while also exhibiting sufficient flexibility to allow deformation to achieve the customized curvature desired.

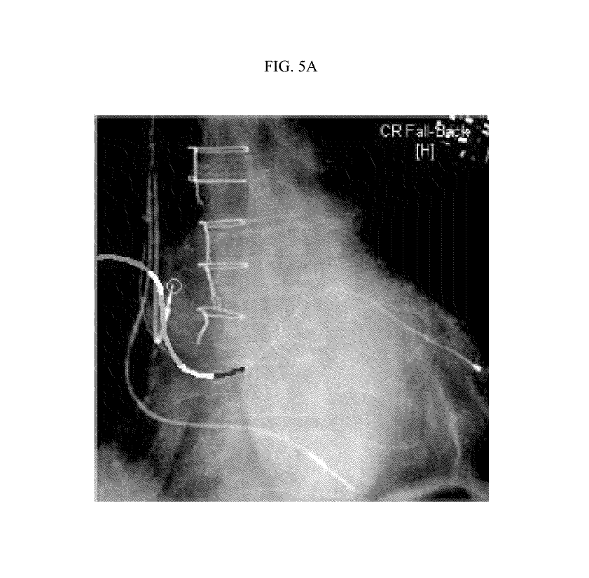

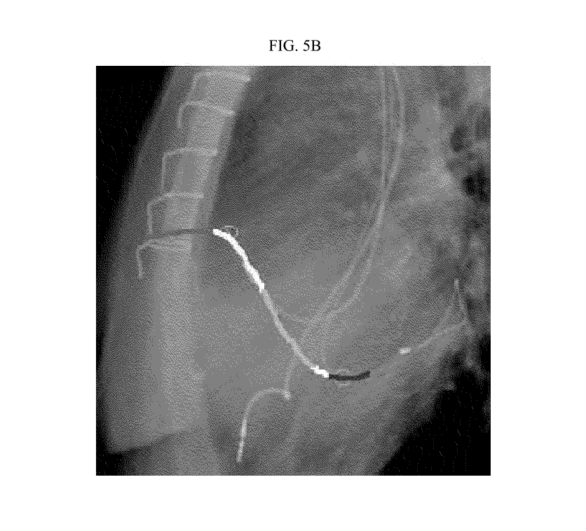

After the CS is cannulated, a balloon catheter and dye is used for CS angiography to identify target branches of the CS. A permanent LV lead is then inserted through the cannula probe, using electrical leads and fluoroscopy. The cannula probe is then stripped away, and the lead is secured, e.g., with an atrial purse-string suture. Finally, the LV lead is tunneled subcutaneously to the left shoulder using an ICD lead tunneler and connected to a subcutaneous or subpectoral generator. RA and RV leads can be inserted by standard endocardial approaches or using RPMS. Accordingly, the disclosed system and method provides improvements in the design, use of active sensors, and insertion by minithoracotomy rather than a transvenous approach. FIGS. 5A-B depict a representative imaging of the disclosed device as deployed within a subject.

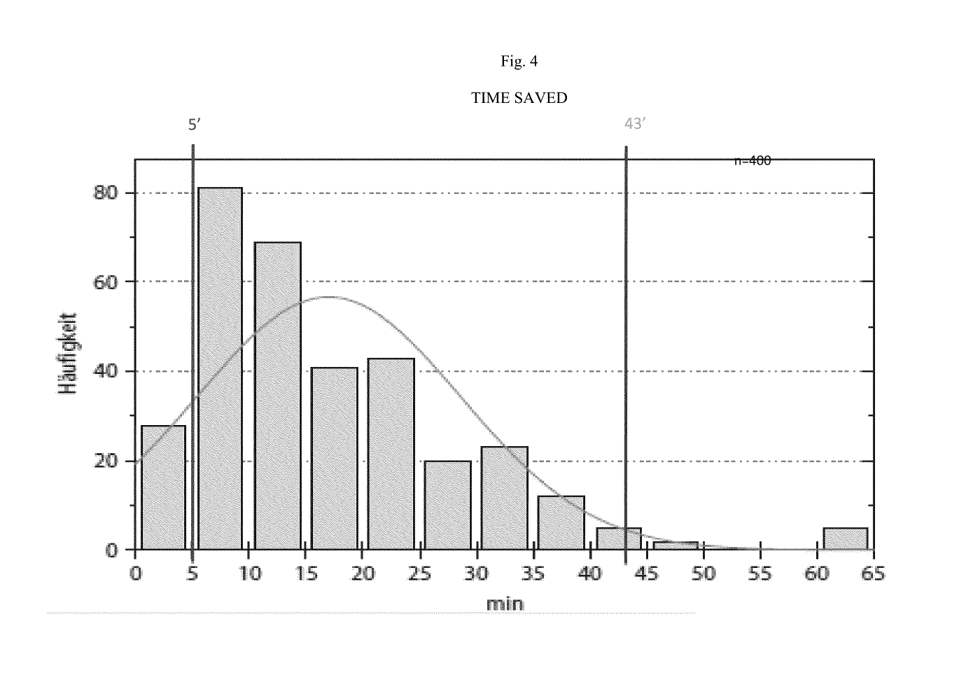

The system and methods disclosed herein is aimed to change clinical practice. RPSM, as disclosed herein, is designed to be superior to any known method of epicardial lead insertion in speed, efficacy, physiologic effect, and limitation of complications. A demonstration of the increased speed of the disclosed system and method is depicted in FIG. 4. These advantages will be accentuated by the presence of intrapericardial adhesions. Common current approaches for epicardial leads require general anesthesia and positive pressure ventilation. If pericardial adhesions are not present, robotic surgery, minithoracotomy or sybxyphoid incisions are applicable. Decubitus positions or cardiac retraction that compromise venous return and cardiac output may be required, however. When dense adhesions are present, a formal thoracotomy is required, which prolongs surgery and increases the risk of blood loss. While RPSM also requires general anesthesia and positive pressure ventilation, compromised venous return, retraction and exposure maneuvers are avoided. Also, the endocardial approach via the CS completely bypasses the need to divide adhesions to expose myocardium.

Surgery with the exemplary cannula probe design is superior to any known method of lead insertion, including current standards, in speed, efficacy, physiologic effect, and incidence of complications. The conventional technique for endocardial insertion is via the cephalic, axillary, or subclavian vein. This is done under local anesthesia, with or without sedation, and invasive incisions into body cavities are not required. However, technical failures occur in 8-13% of attempts, and the incidence of non-responders is as high as 40%. Complications include bleeding, death, infection, lead displacement, coronary sinus dissection, cardiac perforation, and failure to implant a usable LV lead. These risks are mitigated and/or eliminated when employing the devices and techniques disclosed herein.

In some embodiments, the device disclosed herein can be formed from plastic, with a strip-away cannula probe curved in three-dimensional space to enter the CS from a puncture of the right atrial appendage (RAA) wherein the RAA is exposed by right parasternal mediastinotomy (RPMS). The obturator in cannula probe maintains curves, provides lumen for malleable guidewire and sensors to localize CS, if needed. The device includes curvatures in three perpendicular segments. The definition of dimensions, sensors optimization can be defined based on an iterative testing, if so desired. Once CS is entered, CRT is implemented with conventional leads, generator, and tunnelers. The advantages provided by the disclosed system and method include:
Patient Satisfaction—Reduced pain, LOS, morbidity
Physician Satisfaction—Reduced Cost, procedure time, training
Other Benefits: Less short/long term lead failures and reoperations
Applicable to procedures involving CS, intrapericardial (AF, VT ablation), intracardiac (VAD, valve repair) access, transseptal puncture, and to all pacemakers/ICDs.
Family of custom-curved cannula probe-obturators
Software-based selection of optimum curvature
Exchangeable obturators with CS sensors
Malleable obturators allowing curve changes
Soft, flexible outer sheath avoids CS injury
Rapid cannulation<5 min in heart
Zero percent cannulation failure
Lead exchange facilitated
Cardiac retraction avoided
Bleeding risk, surgical pain minimized.

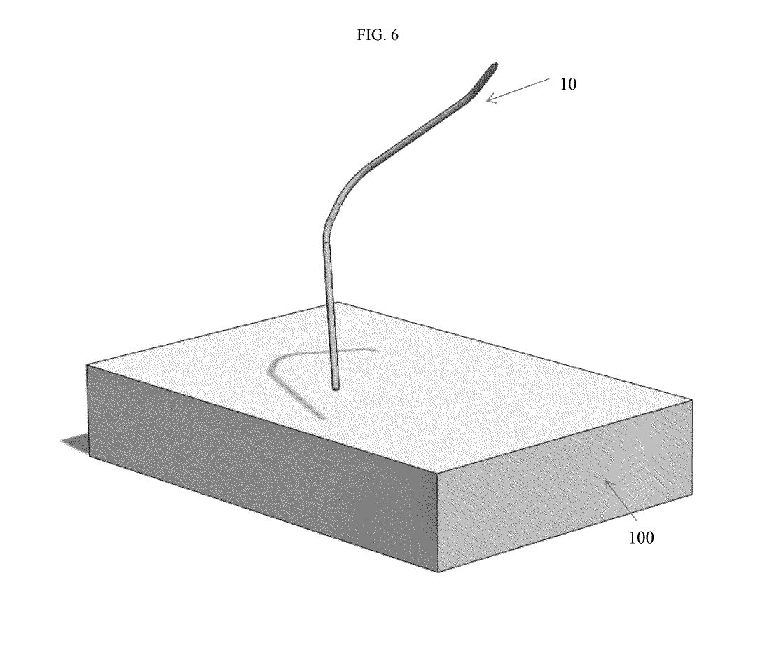

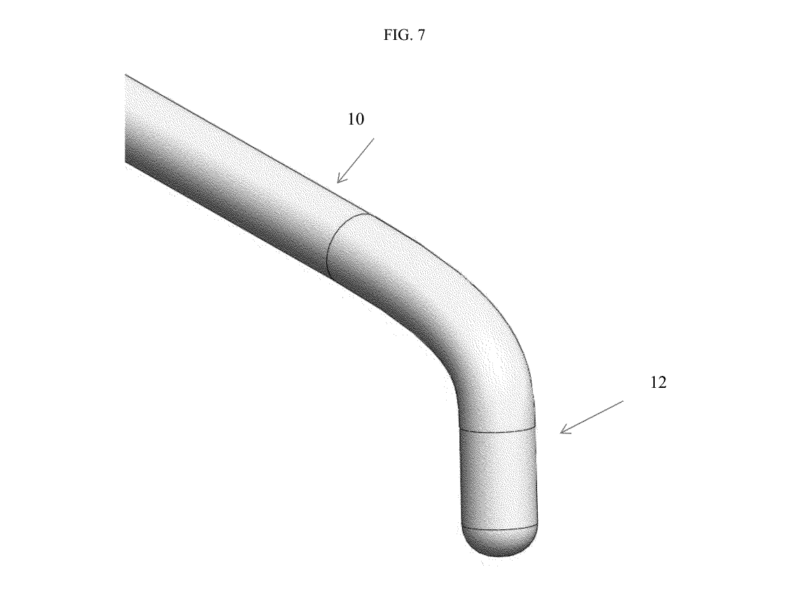

For purpose of illustration and not limitation, an exemplary depiction of the insertion device is provided in FIGS. 6-14. FIG. 6 illustrates an exemplary anatomically shaped CS lead cannula probe 10 having longitudinal body (the shadow projected onto the block 100 is solely provided to help illustrate 3-dimensional curvature which is provided with an optimized contour, as discussed above). FIG. 7 illustrates an exemplary embodiment in which the cannula probe includes a body having a substantially straight longitudinal body and a distal section including a tip configured to form a right-angle relative to the substantially straight longitudinal body of the cannula probe 12. Although the elongated body of the cannula probe is shown having a generally straight profile, this is provided for simplification purposes only. It is to be understood that the probe can have the anatomic curvature along its length discussed above, with a distal end section including a substantially right angle (i.e. 90°) tip relative to the cannula probe body.

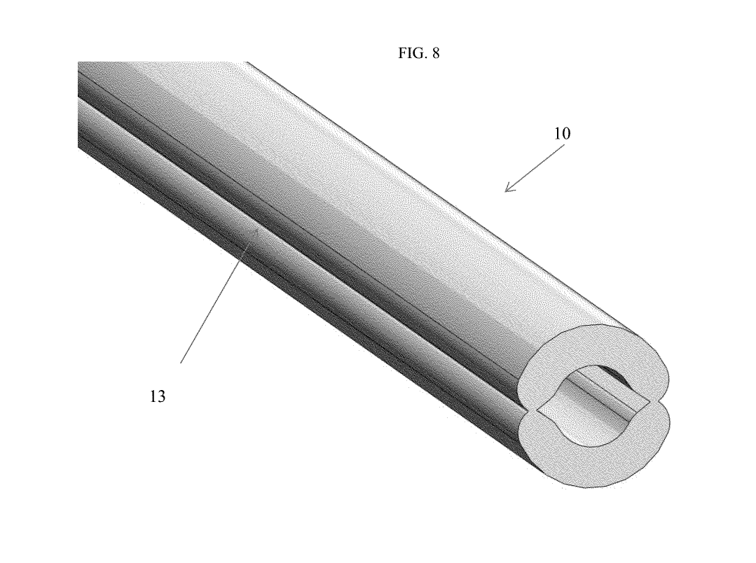

As shown in FIG. 8, the cannula probe 10 can include a line of weakness along a length of the longitudinal body. The line of weakness allows the cannula probe to be split, for example, after delivery to the desired location within the patient. The split can occur along a line of weakness 13. The line of weakness can be formed in the body by including a reduction in material thickness (as shown) between the inner and outer diameters of the wall. Additionally or alternatively, the line of weakness 13 can be formed by a perforation(s) disposed along the longitudinal axis of the probe 10. The perforations (or reduction in thickness profile) can be varied along the length of the probe such that select regions of the probe have a greater concentration of perforations (or greater reduction in thickness) to promote breakage or tearing at different rates along the length of the probe 10. Additionally, the perforations (or reduction in thickness profile) can be configured in a non-linear or patterned fashion.

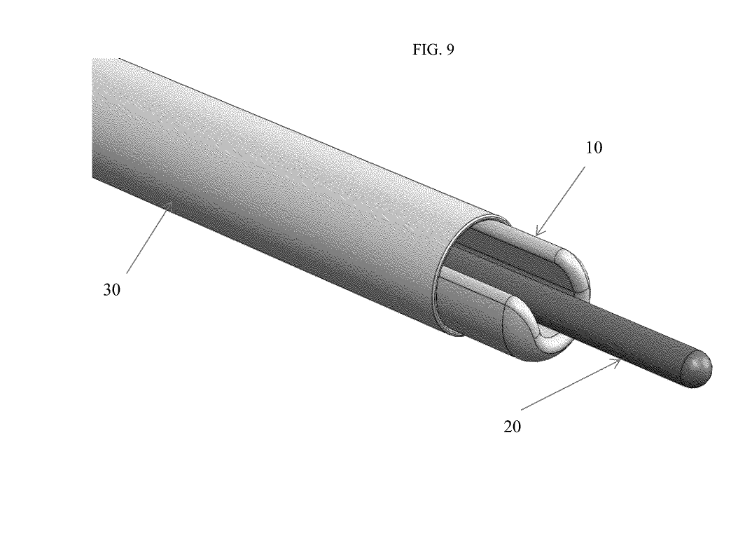

Also, the probe 10 can include a lumen for introducing guide wire 20 through a sheath 30, as shown in FIG. 9. In some embodiments, and as depicted in FIG. 9, the cannula probe 10 does not fully enclose or circumscribe guidewire 20. In operation, the sheath 30 can be retracted to allow separation of probe from guide wire. This separation can occur along the line of weakness 13 (if present) as described above.

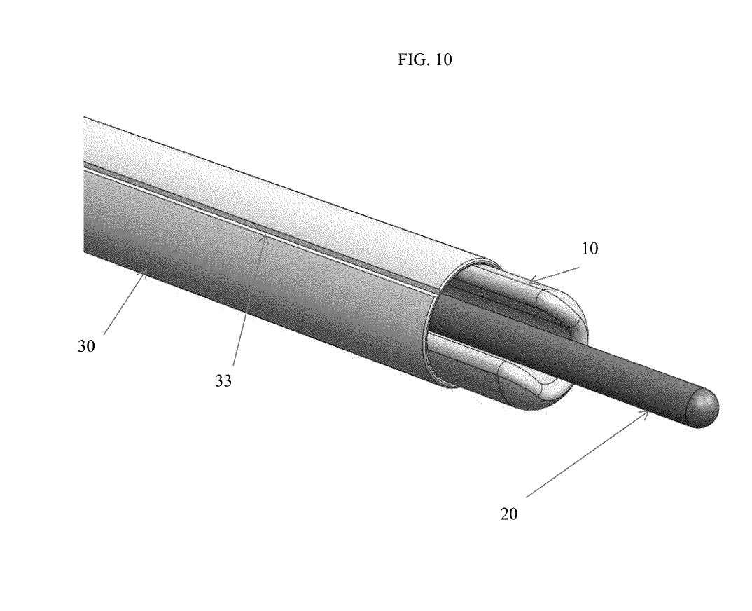

In some embodiments, the sheath 30 can be configured with a line of weakness 33 such that the sheath 30 can be split or severed, as shown in FIG. 10. Similarly to the line of weakness discussed above with respect to the probe 10, the line of weakness in sheath 30 can be configured as a cut, perforation(s) or reduction in wall thickness. Also, the perforations (or reduction in thickness profile) can be varied along the length of the sheath such that select regions of the sheath have a greater concentration of perforations (or greater reduction in thickness) to promote breakage or tearing at different rates along the length of the sheath 30. Additionally, the perforations (or reduction in thickness profile) can be configured in a non-linear or patterned fashion.

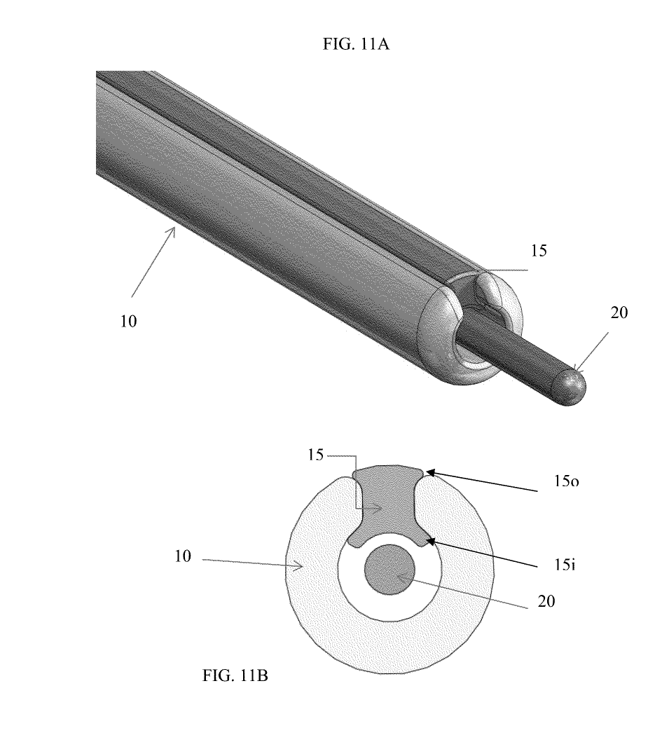

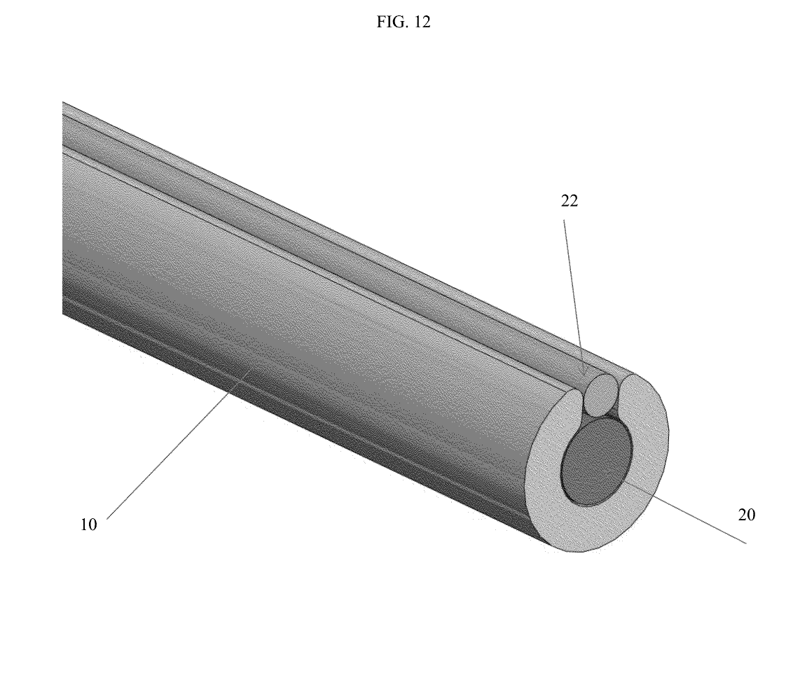

In another embodiment, shown in FIGS. 11A-B, the probe 10 can incorporate a flexible retention piece 15. This flexible retention piece 15 can be configured with a tongue and groove type mating relationship with the wall of the cannula probe 10 such that the retention piece is capable of sliding movement but not radial movement with respect to the probe 10. Accordingly, the retention piece 15 is removed by pulling out along the length of the probe 10. In operation, once the retention piece 15 is removed, the guide wire 20 can be removed. Additionally, following removal of the flexible retention piece 15, a second guide wire 22 may be introduced to aid in the ejection of the targeted guide wire 20, as shown in FIG. 12. As shown in FIG. 11B, the retention piece 15 can be formed with an I-Beam configuration such that the radially inner 15i and outer 150o edges have a larger circumferential width than the middle portion. The radially inner portion 15i of the retention piece 15 can be sized such that it overlies a portion of the inner surface of the probe 10. The greater the amount of overlap, the greater the friction or retention force needed to be overcome in order to remove the retention piece 15. Additionally, while the retention piece 15 is illustrated as a single unitary embodiment that extends the length of probe 10, in some embodiments the retention piece can extend a distance which is less than the longitudinal length of the probe 10, and/or be made of multiple segments.

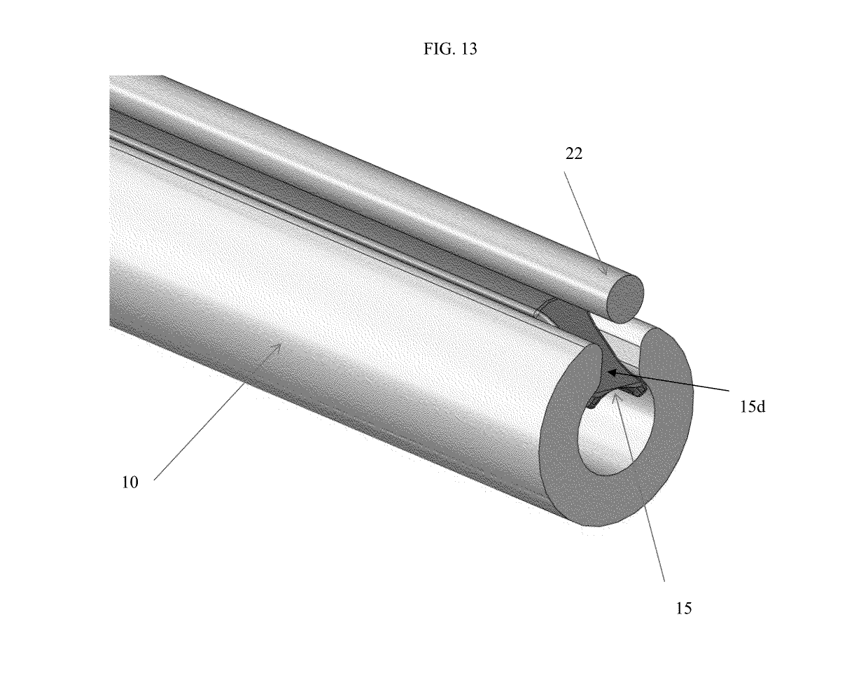

Further, in certain embodiments the flexible retention piece 15 can be configured with a slanted distal end 15d which, when reinserted into the lumen of the probe 10, serves to facilitate ejection of the guide wire 20. As depicted in FIG. 13, the use of such a retention piece completely ejects the guidewire 20 out of the probe 10. Alternatively, the sheath can be constructed to be splittable longitudinally by advancing a splitting device within the central lumen while holding the guidewire to prevent relative movement. The splitting device can include a structural feature (e.g. radially extending protrusion) to facilitate the piercing/splitting of the sheath.

In addition to a standard guide wire, the device of the present disclosure can employ an instrumented guide wire which may be introduced into the probe to measure environmental properties as an indicator of proximity of the probe tip to the coronary sinus. Exemplary sensors include transducers for measuring one or multiple variables including pressure, temperature, and/or oxygen saturation. The sensor(s) can be positioned at the distal tip of the guidewire or cannula. The sensors can be incorporated into the device as an integral unit, or be manufactured separately and assembled to the device.

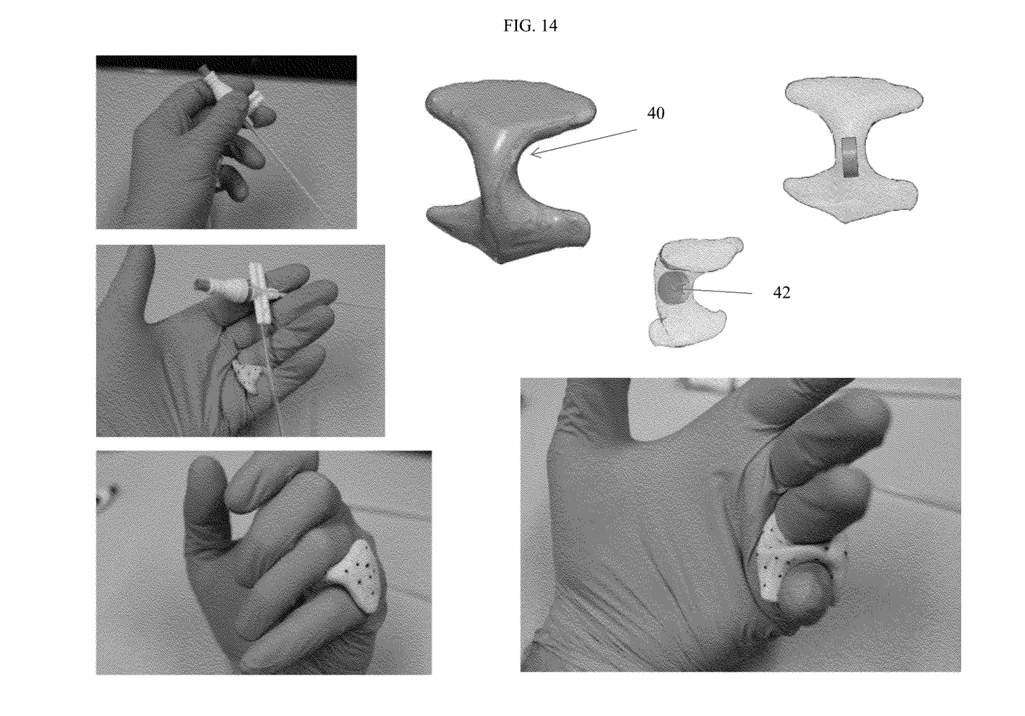

To aid the surgeon in placement of the tip 12, signals from the instrumented probe 10 may be converted into haptic feedback via a vibration motor mounted within an ergonomic casing 40, as shown in FIG. 14. In operation, the amplitude and/or frequency of vibrations exhibited by the casing 40 would change in response to signals from the instrumented probe. In an exemplary embodiment, the motor disposed within a recess 42 in casing 40 and provides tactile feedback to the hand between fingers. In addition to, or instead of, a vibratory response signal, the casing 40 can be configured with an auditory feedback mechanism to alert the operator when the distal end of the probe has been properly inserted within the coronary sinus. The casing is constructed in such a way as to not interfere with dexterity needed during probe positioning. The casing 40 can remain in place without effort (i.e. without squeezing fingers together or curling them). The haptic feedback device could be made wireless by including a battery and wireless transmitter (e.g. Bluetooth or RF). Additionally, it will be understood by artisans of ordinary skill that the device disclosed herein is not necessarily exclusive to the right angle probe.

The device of the present disclosure can be made of biocompatible plastic, such as Polyether ether ketone (PEEK), or in other embodiments, stainless steel. Unlike traditional cannula designs, the device of the present disclosure is formed with sufficient rigidity such that the maximum deformation of the device along its longitudinal length is less than 1.0 mm, in some embodiments. This rigidity retains the right angle shape at the tip during insertion, and transmits mechanical force back to the operator, providing a tactile recognition to the operator of when the probe locates and enters the coronary sinus. Additionally, the hub (not shown) or handle of the device can include calibration marks indicating the direction of the right angle bend relative to the hub, thereby assisting the operator in properly directing the tip. Further, some embodiments are constructed with a non-removable obturator.

An exemplary embodiment of the device is constructed with approximate dimensions of a tubular structure 8 inches in length with an outer diameter of 7 french and channel size accepting a standard flexible metal or plastic guidewire. The right angle bend at the distal tip extends 0.75 inches from the midline of the device. Curvature of the tip is gradual enough to allow atraumatic insertion through a venotomy in the right atrium, e.g. a radius of curvature of 0.75 inches.

In accordance with another aspect of the disclosed subject matter, the devices described herein can be employed for delivery of various materials locally at the CS. For example, the device disclosed herein can be employed as a conduit for injection of cardioplegia during minimally invasive cardiac surgery on cadiopulmonay bypass. Additionally, the devices described herein can be employed for insertion of monitoring instruments into the coronary sinus, e.g., temperature, pressure, oxygen saturation. In other applications, the devices described herein can be employed for insertion of prosthesis including rings, sutures, stiffeners, and pumps to reduce heart failure. By utilizing the RPSM approach disclosed herein, the customized contour of the device allows for a larger diameter catheter to be employed as compared to conventional endocardial techniques. This larger diameter allows for delivery of the various materials described above, while enjoying the benefit of the more accurate placement/delivery afforded by the customized design which provides a delivery port at the CS.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A medical device configured for coronary sinus cannulation comprising:
a cannula probe including a longitudinal body having length, opposing proximal and distal ends, and a lumen extending between the proximal and distal ends,
wherein the longitudinal body includes along its length a contoured section having measurements customized to an anatomy of a particular patient, and the distal end of the longitudinal body includes a tip that forms a substantially right angle relative to the longitudinal body.

2. The medical device of claim 1, wherein the tip is formed from a material having a rigidity sufficient to maintain the substantially right angle orientation.

3. The medical device of claim 1, wherein the maximum deformation of the device along its longitudinal length is less than 1.0 mm.

4. The medical device of claim 1, wherein the substantially right angle at the distal tip extends 0.75 inches from the midline of the device.

5. The medical device of claim 1, wherein the substantially right angle has a radius of curvature of 0.75 inches.

6. The medical device of claim 1, further comprising an outer sheath having a flexible hollow shaft, the cannula probe disposed inside the outer sheath.

7. The medical device of claim 1, wherein a line of weakness extends along at least a portion of the longitudinal length of the cannula probe.

8. The medical device of claim 1, further comprising a guidewire, the guidewire disposed within the lumen of the cannula probe.

9. The medical device of claim 8, wherein the cannula probe circumferentially extends a distance less than the circumference of the guidewire.

10. The medical device of claim 8, further comprising a retention piece, the retention piece slidingly received by the cannula probe.

11. The medical device of claim 10, wherein the retention piece is configured with a slanted distal end.

12. The medical device of claim 11, wherein the slanted distal end of the retention piece radially displaces the guidewire out of the cannula probe lumen upon axial movement of the retention piece.

13. The medical device of claim 10, wherein the retention piece has radially inner and outer edges with a larger circumferential width than a middle portion disposed between the radially inner and outer edges.

14. The medical device of claim 12, wherein the radially inner portion of the retention piece overlies a portion of an inner surface of the cannula probe.

15. The medical device of claim 12, wherein the radially inner outer edge of the retention piece is circumferentially aligned with an outer surface of the cannula probe.

16. The medical device of claim 1, further comprising at least one sensor, the sensor disposed at the distal tip of the cannula probe.

17. The medical device of claim 8, further comprising at least one sensor, the sensor disposed at the distal tip of the guidewire.

18. A medical kit comprising:
a cannula probe including a longitudinal body having a length, opposing proximal and distal ends, and a lumen extending between the proximal and distal ends, wherein the longitudinal body includes along its length a contoured section having measurements customized to an anatomy of a particular patient, and the distal end of the longitudinal body includes a tip that forms a substantially right angle relative to the longitudinal body; and
a haptic feedback device, the haptic feedback device configured to receive a signal from the sensor.

19. The kit of claim 18, wherein the haptic feedback device includes a vibratory feedback mechanism.

20. The kit of claim 18, wherein the haptic feedback device includes an auditory feedback mechanism.

21. The kit of claim 18, wherein the haptic feedback device and cannula probe are configured as separate components.

22. The kit of claim 18, wherein the haptic feedback device includes a transmitter for wireless communication with the sensor disposed at the distal tip of the cannula probe.

23. A method of manufacturing a cannula probe customized to a particular patient, the method comprising:
obtaining measurement data correlating to a patient's entry ribcage, entry RAA, curve bias, entry coronary sinus, inside coronary sinus, or a combination thereof, and
fabricating a cannula probe having a customized geometry based on the obtained measurement data.

23. The method of claim 23, wherein the measurement data is obtained by a CT scan of the patient's anatomy.

24. The method of claim 24, wherein the data from the CT scan is converted into 3D data.

25. The method of claim 25, wherein the method further includes preparing a physical model of the patient's anatomy and an anatomical pathway to the coronary sinus is extracted.

26. The method of claim 26, wherein the cannula probe has a customized geometry based on the extracted pathway to the coronary sinus of the patient.

27. A method of treating a patient by cannulating the coronary sinus of the heart from a right side insertion after the patient has failed endocardial lead insertion, the method comprising:
selecting a cannula probe including a longitudinal body having a length, opposing proximal and distal ends, and a lumen extending between the proximal and distal ends, wherein the longitudinal body has a geometry along its length that is customized to the anatomy of the treated patient.

28. The method of treating of claim 27, wherein the longitudinal body includes along its length a contoured section having measurements customized to the patient's anatomy, and the distal end of the longitudinal body includes a tip having a length that forms a substantially right angle relative to the longitudinal body.

29. The method of claim 28, wherein the tip length is preformed having the substantially right angle orientation.

30. The method of claim 27, wherein the geometry of the cannula probe is derived from measurements of the patient's entry ribcage, entry RAA, curve bias, entry coronary sinus, inside coronary sinus, or a combination thereof.
